(19) United States
(12) Patent Application Publication
(10) Pub. No.: US 2016/0046603 A1
(43) Pub. Date: Feb. 18, 2016

(54) Crystalline Forms of D-Glucitol, 1-Deoxy-1-(Methylamino)-, 1-(6-Amino-3,5-Difluoropyridine-2-Yl)-8-Chloro-6-Fluoro-1,4-Dihydro-7-(3-Hydroxyazetidin-1-Yl)-4-Oxo-3-Quinolinecarboxylate

(71) Applicant: MELINTA THERAPEUTICS, INC., New Haven, CT (US)

(72) Inventors: Roger HANSELMANN, Branford, CT (US); Maxwell M. REEVE, Guilford, CT (US)

(21) Appl. No.: 14/773,655

(22) PCT Filed: Mar. 7, 2014
(86) PCT No.: PCT/US14/21946

§ 371 Date: Sep. 8, 2015

Related U.S. Application Data
(60) Provisional application No. 61/775,089, filed on Mar. 8, 2013.

(51) Int. Cl. C07D 401/14

(52) U.S. Cl. CPC C07D 401/14; C07B 2200/13

(57) ABSTRACT

The present disclosure relates generally to crystalline forms of anhydrous D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1- yl)-4-oxo-3-quinolinecarboyxlate, compositions comprising the same, and methods of making the same. Delafloxacin is an fluoroquinolone antibiotic with the chemical structure and the chemical name 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate. Studies have indicated the existence of three anhydrous polymorphs of delafloxacin, as well as a trihydrate and methanol and ethanol solvates.

### FIELD OF THE INVENTION

The present disclosure relates generally to crystalline forms of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate, compositions comprising the same, and methods of making the same.

### BACKGROUND

Delafloxacin is an fluoroquinolone antibiotic with the chemical structure

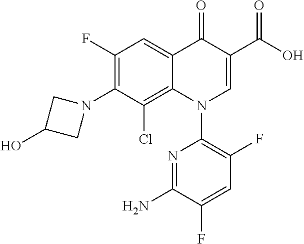 [SMILES: Nc1nc(-n2cc(C(=O)O)c(=O)c3cc(F)c(N4CC(O)C4)c(Cl)c32)c(F)cc1F]

and the chemical name 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate. Studies have indicated the existence of three anhydrous polymorphs of delafloxacin, as well as a trihydrate and methanol and ethanol solvates. Delafloxacin has been shown to be highly potent against both Gram-negative and Gram-negative bacteria, with a balanced inhibition of both topoisomerase and gyrase enzyme targets. The most stable polymorphic form, Form 1, is generated by vacuum drying of the trihydrate, and is currently being pursued in clinical trials for treatment of bacterial infections. Phase 2 trials have shown delafloxacin to be successful in both IV and oral dosage forms, with good tolerance and safety demonstrated in nearly 1,400 patients.

The meglumine (N-methyl-D-glucamine) salt of delafloxacin demonstrates several advantageous properties over the parent acid, such as improved solubility, dissolution and bioavailability.

Crystallinity of drugs affects, among other physical and mechanical properties, the drug's ease of preparation, stability, ease of formulation, solubility, dissolution rate, hardness, compressibility and melting point. Polymorphic forms occur where the same composition of matter crystallizes in a different lattice arrangement resulting in different thermodynamic properties and stabilities specific to the particular polymorph form. Different polymorphs of a given compound may differ from each other with respect to one more physical properties, such as solubility and dissociation, density, crystal shape, compaction behavior, flow properties, and/or solid state stability. In cases where two or more polymorph substances can be produced, it is desirable to have a method to make each form in pure form. In deciding which polymorph is preferable in a given situation, the numerous properties of the polymorphs must be compared and the preferred polymorph chosen based on the many physical property variables. Because these properties and considerations may, in turn, affect a drug's manufacture and their in vivo pharmacological utility, there is an existing need in the chemical and therapeutic arts for identification of crystalline polymorphic forms of drugs, including delafloxacin, and ways of reproducibly making them.

### BRIEF DESCRIPTION OF THE DRAWINGS

FIG. 1 shows an X-ray powder diffraction (XRPD) pattern of crystalline anhydrous Form 1A D-glucitol, 1-deoxy-1-(methylamino)-, 1-(6-amino-3,5-difluoropyridin-2-yl)-8-chloro-6-fluoro-1,4-dihydro-7-(3- hydroxyazetidin-1-yl)-4-oxo-3-quinolinecarboyxlate.

FIG. 2 shows an X-ray powder diffraction (XRPD) pattern of crystalline anhydrous Form 1B D-glucitol, 1-deoxy-1-(methylamino)-, 1-(6-amino-3,5-difluoropyridin-2-yl)-8-chloro-6-fluoro-1,4-dihydro-7-(3- hydroxyazetidin-1-yl)-4-oxo-3-quinolinecarboyxlate.

FIG. 3 shows a Modulated Differential Scanning Calorimetry (mDSC) thermogram of crystalline anhydrous Form 1A D-glucitol, 1-deoxy-1-(methylamino)-, 1-(6-amino-3,5-difluoropyridin-2-yl)-8-chloro-6-fluoro-1,4-dihydro- 7-(3-hydroxyazetidin-1-yl)-4-oxo-3-quinolinecarboyxlate.

FIG. 4 shows a Modulated Differential Scanning Calorimetry (mDSC) thermogram of crystalline anhydrous Form 1B D-glucitol, 1-deoxy-1-(methylamino)-, 1-(6- amino-3,5-difluoropyridin-2-yl)-8-chloro-6-fluoro-1,4-dihydro-7-(3-hydroxyazetidin-1-yl)-4-oxo-3-quinolinecarboyxlate.

FIG. 5 shows overlayed XRPD diffraction patterns of crystalline anhydrous D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate.

FIG. 6 shows overlayed XRPD diffraction patterns of crystalline anhydrous Form 1A and crystalline anhydrous Form 1B of D-glucitol, 1-deoxy-1-(methylamino)-, 1-(6-amino-3,5-difluoropyridin-2-yl)-8-chloro-6-fluoro-1,4- dihydro-7-(3-hydroxyazetidin-1-yl)-4-oxo-3-quinolinecarboyxlate.

### SUMMARY

The present disclosure relates generally to crystalline forms of anhydrous D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate, compositions comprising the same, methods of making the same, and methods of using the same to treat bacterial infections.

In producing industrial scale batches of delafloxacin meglumine anhydrate, the inventors have unexpectedly discovered that what was thought to be a single crystalline form was actually two distinct polymorphs with different properties. These polymorphs, designated Form 1A and Form 1B, are formed during the final salt forming and dehydration steps in the synthesis of delafloxacin meglumine anhydrate. The inventors have further discovered conditions for controlling which polymorph is formed, and conditions for converting Form 1B to Form 1A.

The inventors have characterized the identified Form 1A and Form 1B polymorphic forms of anhydrous delafloxacin meglumine, and developed conditions for controlling which form is produced. This discovery has allowed for the production of a consistent crystalline form of anhydrous delafloxacin meglumine which can be used in clinical trials and sold as a commercial product upon approval.

In one aspect, a crystalline anhydrous Form 1A of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate is disclosed herein.

In another aspect, a crystalline anhydrous Form 1B of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate is disclosed herein.

In another aspect, a pharmaceutical composition is disclosed herein comprising the crystalline anhydrous forms or compositions disclosed herein and a pharmaceutically acceptable carrier or excipient.

In another aspect, a method of treating a bacterial infection in a fish or mammal in need thereof is disclosed herein, the method comprising administering to the fish or mammal a therapeutically effective amount of a composition comprising the crystalline anhydrous form, composition or pharmaceutical composition disclosed herein.

In another aspect, a process for the preparation of a crystalline anhydrous Form 1A of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1- yl)-4-oxo-3-quinolinecarboyxlate is disclosed herein, which process comprises the steps of: (a) drying delafloxacin meglumine trihydrate; and (b) exposing the dried delafloxacin meglumine to heat and humidity.

In another aspect, a process for the preparation of a crystalline anhydrous Form 1B of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1- yl)-4-oxo-3-quinolinecarboyxlateis disclosed herein, which process comprises drying delafloxacin meglumine trihydrate under low humidity conditions.

In another aspect, a process for producing a crystalline anhydrous Form 1A of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate from a crystalline Form 1B of D-glucitol, 1-deoxy-1-(methylamino)-, 1-(6-amino-3,5-difluoropyridin-2-yl)-8-chloro-6-fluoro-1,4-dihydro-7-(3- hydroxyazetidin-1-yl)-4-oxo-3-quinolinecarboyxlate is disclosed herein, the process comprising exposing delafloxacin meglumine to heat and humidity.

In another aspect, crystalline anhydrous forms prepared by the processed disclosed herein are disclosed herein.

### DETAILED DESCRIPTION

Unless otherwise defined, all technical and scientific terms used herein have the same meaning as commonly understood by one of ordinary skill in the art to which this disclosure belongs. Although methods and materials similar or equivalent to those described herein can be used in accordance with the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. Other features and advantages of the present disclosure will be apparent from the following detailed description, and from the claims.

As discussed above, the inventors have unexpectedly discovered two different polymorphs formed during the final salt forming step and dehydration of delafloxacin meglumine, designated Form 1A and Form 1B, and have discovered conditions for controlling which polymorph is formed. Further, it was discovered that Form 1A displayed improved dissolution characteristics and thermodynamic stability over Form 1B, and that Form 1B was metastable and could transform to Form 1A under certain storage conditions. Due to these distinct characteristics, the inventors developed methods for reliably producing either Form 1A or Form 1B, and methods for converting Form 1B to Form 1A.

International Patent Application Publication No. WO 2006/042034 describes the meglumine salt of delafloxacin, both in anhydrous and trihydrate form. However, this publication does not disclose the multiple polymorphic forms identified by the inventors or the different characteristics and properties thereof. Importantly, the prior art does not disclose the process developed by the inventors for selectively preparing each of the identified polymorphic forms, and for converting Form 1B to Form 1A.

Table 1, below lists certain peaks identified by the inventors in XRPD experiments which demonstrate the differences between the identified polymorphic forms. The data below was obtained from a copper radiation source (Cu—Kα, 40 kV, 4 mA).

TABLE 1

| | |
|---|---|
| Peak Position | |
| (2-Theta) | Shift |

| Form 1B | Form 1A | (2-Theta) |
|---|---|---|
| 6.30 | 6.35 | −0.05 |
| 12.58 | 12.70 | −0.12 |
| 18.90 | 19.10 | −0.20 |
| 20.34 | 20.50 | −0.16 |

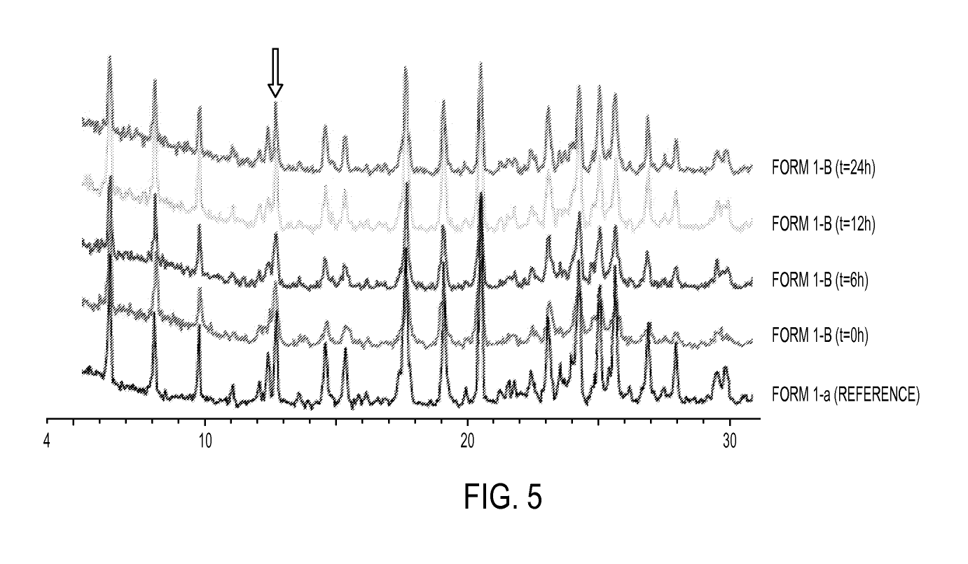

The inventors have also discovered that crystalline anhydrous Form 1B delafloxacin meglumine converts to crystalline anhydrous Form 1A delafloxacin meglumine on exposure to humidity and heat under specific conditions. This process can be followed by monitoring changes in the b-axis reflections for unit cell dimensions determined by XRPD, as shown in Table 1 and FIG. 5. Transformation to the more thermodynamically stable shorter b-axis morphology is irreversibly mediated by either heat or moisture. However, excessive moisture can also convert the material back to the trihydrate. The inventors have discovered conditions which provide for reliable conversion to crystalline anhydrous Form 1A delafloxacin meglumine. Batches of up to 90 kg of crystalline anhydrous Form 1A delafloxacin meglumine have been processed using the process disclosed herein.

### A. Definitions

The content of any publication cited herein is incorporated by reference.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “crystalline,” as used herein, means having a regularly repeating arrangement of molecules or external face planes.

The term “substantial crystalline purity,” as used herein, means at least about 90% crystalline purity.

The term “crystalline purity,” as used herein, means percentage of a crystalline compound in a sample which may contain an amorphous form of the same compound, at least one other crystalline form of the compound or a mixture thereof.

Unless stated otherwise, percentages stated throughout this specification are weight/weight (w/w) percentages.

The term “amorphous,” as used herein, means essentially without regularly repeating arrangement of molecules or external face planes.

The term “mixture,” as used herein, means a combination of at least two substances, in which one substance may be completely soluble, partially soluble or essentially insoluble in the other substance.

The term “solvent,” as used herein, means a substance, preferably a liquid or a miscible, partially miscible or immiscible mixture of two or more liquids, which is capable of completely dissolving, partially dissolving, dispersing or partially dispersing another substance, preferably a solid or a mixture of solids.

The term “anti-solvent,” as used herein, means a solvent in which a compound is essentially insoluble.

It is meant to be understood that, because many solvents and anti-solvents contain impurities, the level of impurities in solvents and anti-solvents for the practice of this disclosure, if present, are at a low enough concentration that they do not interfere with the intended use of the solvent in which they are present.

It is meant to be understood that peak heights in a powder x-ray diffraction pattern may vary and will be dependent on variables such as the temperature, crystal size, crystal habit, sample preparation or sample height in the analysis well of the Scintag×2 Diffraction Pattern System.

It is also meant to be understood that peak positions may vary when measured with different radiation sources. For example, Cu—Kα₁, Mo—Kα, Co—Kα and Fe—Kα radiation, having wavelengths of 1.54060 Å, 0.7107 Å, 1.7902 Å and 1.9373 Å, respectively, may provide peak positions which differ from those measured with Cu—Kα radiation.

In some embodiments, the compositions disclosed herein are incorporated into a pharmaceutical composition or medicament.

The therapeutically effective amount of a crystalline compound disclosed herein depends on recipient of treatment (age, body weight, sex and general health), the biological activity of the particular preparation, the disorder being treated and severity thereof, composition containing it, time of administration, route of administration, duration of treatment, its potency, its rate of clearance and whether or not another drug is co-administered. The amount of a crystalline compound disclosed herein used to make a composition to be administered daily to a patient in a single dose or in divided doses is from about 0.03 to about 200 mg/kg body weight. Single dose compositions contain these amounts or a combination of submultiples thereof.

In some embodiments, the compositions disclosed herein comprise at least one pharmaceutically acceptable vehicle, diluent, excipient, carrier, or combination thereof. In some embodiments, the composition comprises a pharmaceutically acceptable vehicle selected from saline, sterile water, Ringer's solution, isotonic sodium chloride solutions and mixtures thereof. In some embodiments, the compositions disclosed herein comprise one or more components selected from adjuvants, flavorings, colorants, wetting agents, emulsifying agents, pH buffering agents, preservatives and combinations of thereof

In some embodiments, the compositions disclosed herein are administered by a method selected from orally, rectally, or parenterally (e.g., intramuscular, intravenous, subcutaneous, nasal or topical). In some embodiments, the form in which the compositions are administered will be determined by the route of administration. In some embodiments, the compositions comprise capsular or tablet formulations (such as for oral and rectal administration), liquid formulations (such as for oral, intravenous, intramuscular, subcutaneous, ocular, intranasal, inhalation-based and transdermal administration) and slow releasing microcarriers (such as for rectal, intramuscular or intravenous administration).

In some embodiments, the compositions disclosed herein are administered bucally, ophthalmically, orally, osmotically, parenterally (intramuscularly, intrasternally, intravenously, subcutaneously), rectally, topically, transdermally or vaginally.

In some embodiments, the compositions disclosed herein are ophthalmically administered dosage forms administered as elixirs, emulsions, microemulsions, ointments, solutions, suspensions or syrups.

In some embodiments, the compositions disclosed herein are orally administered solid dosage forms administered as capsules, dragées, emulsions, granules, pills, powders, solutions, suspensions, tablets, microemulsions, elixirs, syrups or powders for reconstitution.

In some embodiments, the compositions disclosed herein are osmotically or topically administered dosage forms administered as creams, gels, inhalants, lotions, ointments, pastes or powders.

In some embodiments, the compositions disclosed herein are parenterally administered dosage forms administered as aqueous or oleaginous suspensions.

In some embodiments, the compositions disclosed herein are rectally or vaginally administered dosage forms administered as creams, gels, lotions, ointments or pastes.

In some embodiments, the compositions disclosed herein further comprise one or more excipients. In some embodiments, the excipient is selected from encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof.

In some embodiments, the compositions disclosed herein comprise components disclosed in Handbook of Pharmaceutical Excipients, Fifth Edition, Eds. R. C. Rowe, et al., Pharmaceutical Press (2006); Remington's Pharmaceutical Sciences, 18th ed. (Mack Publishing Company, 1990); and Remington: The Science and Practice of Pharmacy, 20th Edition, Baltimore, Md.: Lippincott Williams & Wilkins, 2000, which are incorporated by reference herein in their entirety.

In some embodiments, compositions of the present disclosure are to be administered orally in solid dosage form and include one or more excipients selected from agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

In some embodiments, compositions of the present disclosure are to be administered ophthalmically or orally in liquid dosage and include one or more excipients selected from 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water and mixtures thereof.

In some embodiments, compositions of the present disclosure are to be administered osmotically and include one or more excipients selected from chlorofluorohydrocarbons, ethanol, water and mixtures thereof.

In some embodiments, the compositions of the present disclosure are to be administered parenterally and include one or more excipients selected from 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, USP or isotonic sodium chloride solution, water and mixtures thereof.

In some embodiments, the compositions of the present disclosure are to be administered rectally or vaginally and include one or more excipients selected from cocoa butter, polyethylene glycol, wax and mixtures thereof.

In some embodiments, compositions of the present disclosure include carriers, excipients, and diluents. In some embodiments, the carriers, excipients and diluents are selected from lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl and propylhydroxybenzoates, talc, magnesium stearate, mineral oil and combinations thereof. In some embodiments, the pharmaceutical compositions disclosed herein include lubricating agents, wetting agents, emulsifying agents, suspending agents, preserving agents, sweetening agents, flavoring agents or mixtures thereof.

In some embodiments, the pharmaceutical compositions disclosed herein are formulated so as to provide quick, sustained or delayed release of the active ingredient after administration to the patient.

### B. Embodiments

The present disclosure relates generally to crystalline anhydrous forms of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate, compositions comprising the same, and methods of making the same.

In one aspect, a crystalline anhydrous Form 1A of D-glucitol, 1-deoxy-1- (methylamino)-, 1-(6-amino-3,5-difluoropyridin-2-yl)-8-chloro-6-fluoro-1,4-dihydro-7-(3-hydroxyazetidin-1-yl)-4-oxo-3- quinolinecarboyxlate is disclosed herein.

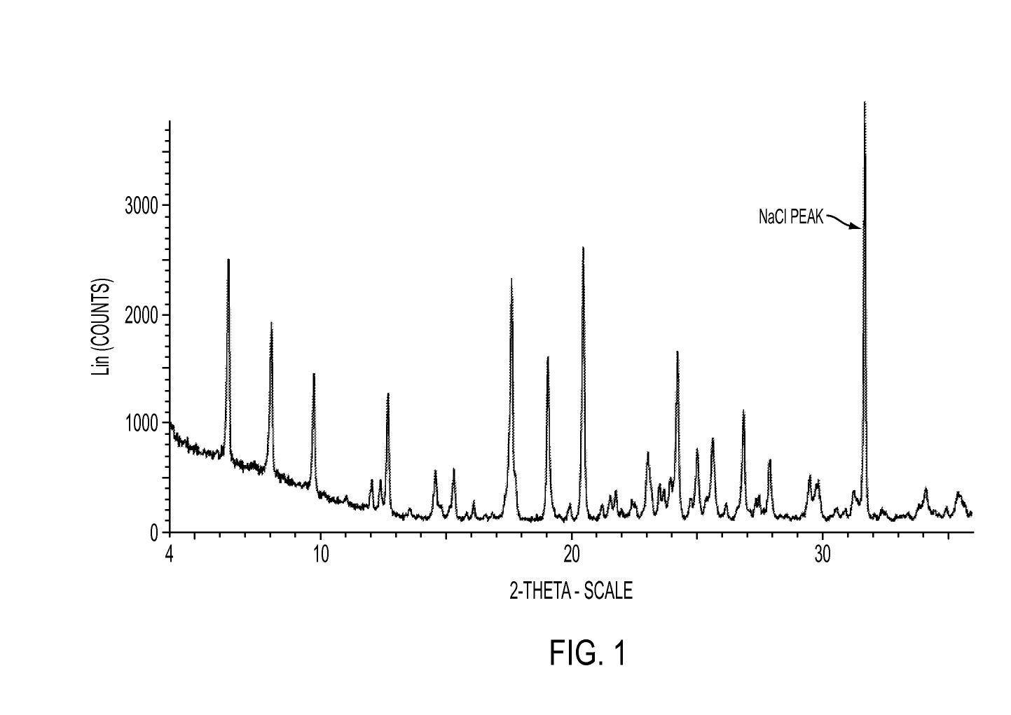

In some embodiments, the crystalline anhydrous forms disclosed herein are characterized by an X-ray powder diffraction pattern substantially in accordance with that shown in FIG. 1, wherein the pattern is obtained from a copper radiation source (Cu—Kα, 40 kV, 4 mA).

In some embodiments, the crystalline anhydrous forms disclosed herein are characterized by an X-ray powder diffraction pattern having peaks at about 6.35, 12.70, 19.10 and 20.50 degrees 2θ, wherein the pattern is obtained from a copper radiation source (Cu—Kα, 40 kV, 4 mA).

In another aspect, a crystalline anhydrous Form 1B of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate is disclosed herein.

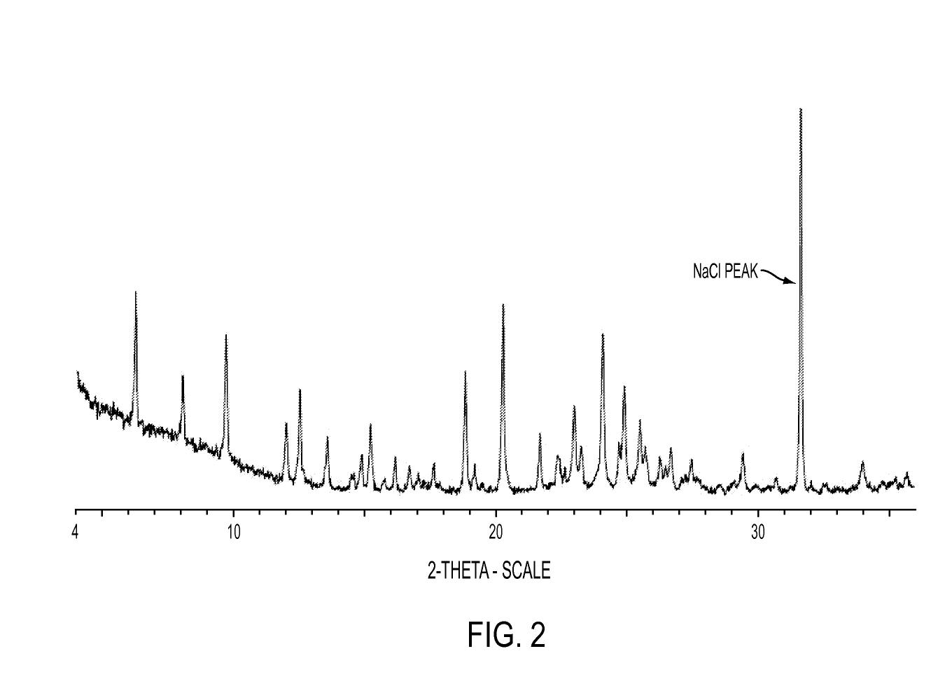

In some embodiments, the crystalline anhydrous forms disclosed herein are characterized by an X-ray powder diffraction pattern substantially in accordance with that shown in FIG. 2, wherein the pattern is obtained from a copper radiation source (Cu—Kα, 40 kV, 4 mA).

In some embodiments, the crystalline anhydrous forms disclosed herein are characterized by an X-ray powder diffraction pattern having peaks at about 6.30, 12.58, 18.90 and 20.34 degrees 2θ, wherein the pattern is obtained from a copper radiation source (Cu—Kα, 40 kV, 4 mA).

In some embodiments, the crystalline anhydrous forms disclosed herein are characterized by a melting point of about 168-171° C.

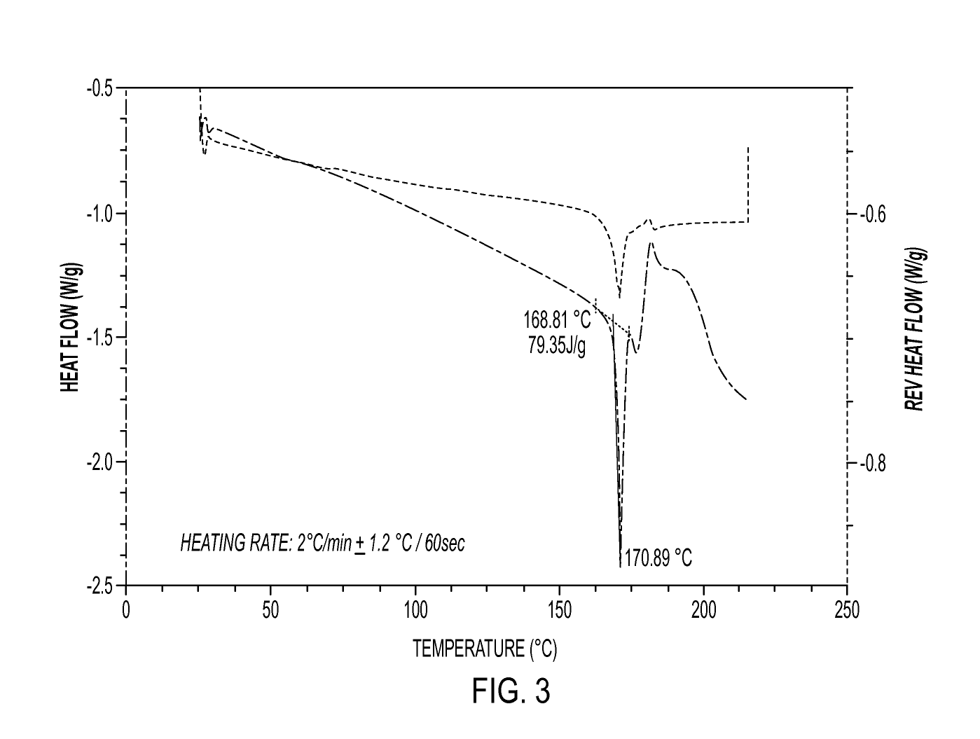

In some embodiments, the crystalline anhydrous forms disclosed herein are characterized by the differential scanning calorimetry thermogram shown in FIG. 3.

In some embodiments, the crystalline anhydrous forms disclosed herein are characterized by a melting point of about 168-171° C. In some embodiments, the crystalline forms disclosed herein are further characterized by an exothermic transition at about 93° C. to about 99° C.

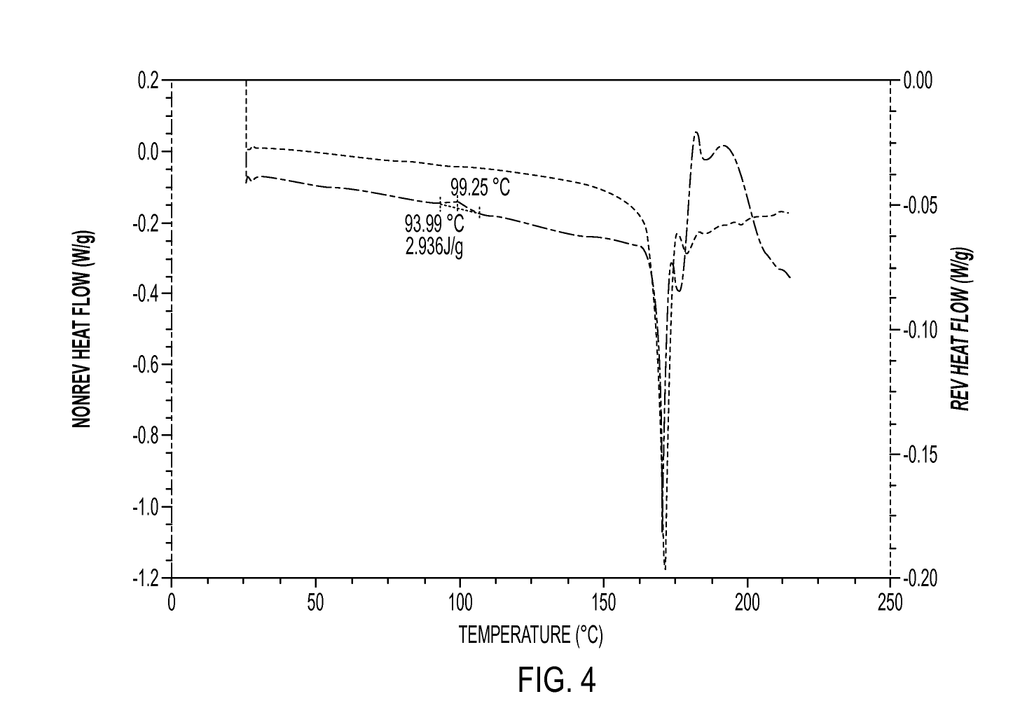

In some embodiments, the crystalline anhydrous forms disclosed herein are characterized by the differential scanning calorimetry thermogram shown in FIG. 4.

In some embodiments, the compositions disclosed herein comprise less than about 10% of a crystalline anhydrous Form 1B delafloxacin meglumine. In some embodiments, the compositions disclosed herein comprise less than about 5% of crystalline anhydrous Form 1B delafloxacin meglumine. In some embodiments, the compositions disclosed herein comprise less than about 3% of crystalline anhydrous Form 1B delafloxacin meglumine. In some embodiments, the compositions disclosed herein comprise less than about 2% of crystalline anhydrous Form 1B delafloxacin meglumine. In some embodiments, the compositions disclosed herein comprise less than about 1% of crystalline anhydrous Form 1B delafloxacin meglumine.

In some embodiments, the compositions disclosed herein are characterized by an X-ray powder diffraction pattern substantially lacking peaks at about 6.30, 12.58, 18.90 and 20.34 degrees 2θ, wherein the pattern is obtained from a copper radiation source (Cu—Kα, 40 kV, 4 mA).

In some embodiments, the compositions disclosed herein comprise less than about 10% of a crystalline anhydrous Form 1A delafloxacin meglumine. In some embodiments, the compositions disclosed herein comprise less than about 5% of a crystalline anhydrous Form 1A delafloxacin meglumine. In some embodiments, the compositions disclosed herein comprise less than about 3% of a crystalline anhydrous Form 1A delafloxacin meglumine. In some embodiments, the compositions disclosed herein comprise less than about 2% of a crystalline anhydrous Form 1A delafloxacin meglumine. In some embodiments, the compositions disclosed herein comprise less than about 1% of a crystalline Form 1A delafloxacin meglumine.

In some embodiments, the compositions disclosed herein are characterized by an X-ray powder diffraction pattern substantially lacking peaks at about 6.35, 12.70, 19.10 and 20.50 degrees 2θ, wherein the pattern is obtained from a copper radiation source (Cu—Kα, 40 kV, 4 mA).

In some embodiments, the crystalline anhydrous forms disclosed herein have substantially crystalline purity. In some embodiments, the crystalline anhydrous forms disclosed herein have at least about 90% crystalline purity.

In another aspect, pharmaceutical compositions are disclosed which comprise the crystalline anhydrous forms or compositions disclosed herein and a pharmaceutically acceptable carrier or excipient. In some embodiments, the pharmaceutical compositions are an oral dosage forms. In some embodiments, the pharmaceutical compositions are in the form of a tablet, capsule, lozenge, powder, syrup, suspension, ointment or dragée.

In another aspect, methods of treating a bacterial infection in a fish or mammal in need thereof are disclosed herein, the methods comprising administering to the fish or mammal a therapeutically effective amount of a composition comprising the crystalline anhydrous forms, compositions or pharmaceutical compositions disclosed herein. In some embodiments, the compositions are administered to a mammal. In some embodiments, the therapeutically effective amount is from about 0.03 to about 200 mg/kg body weight.

In another aspect, processes for the preparation of a crystalline anhydrous Form 1A of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate are disclosed herein, which processes comprise the steps of: (a) drying delafloxacin meglumine trihydrate; and (b) exposing the dried delafloxacin meglumine to heat and humidity.

In some embodiments, said drying delafloxacin meglumine trihydrate is performed under reduced pressure.

In some embodiments, said drying delafloxacin meglumine trihydrate is performed at a temperature between about 30° C. and about 60° C.

In some embodiments, the delafloxacin meglumine trihydrate is dried for between about 24 hours to about 72 hours.

In some embodiments, the delafloxacin meglumine trihydrate is dried for about 48 hours.

In some embodiments, the heat is between about 30° C. and about 70° C. In some embodiments, the heat is between about 50° C. and about 60° C.

In some embodiments, the humidity is between about 30% and about 70% relative humidity. In some embodiments, the humidity is between about 40% and about 60% relative humidity.

In some embodiments, the dried delafloxacin meglumine is exposed to heat and humidity for between about 8 hours to about 36 hours.

In some embodiments, the dried delafloxacin meglumine is exposed to heat and humidity for about 18 hours.

In some embodiments, the process further comprises drying the delafloxacin meglumine which has been exposed to heat and humidity to further drying. In some embodiments, said further drying comprise drying at a temperature between about 30° C. and about 70° C. In some embodiments, said further drying comprise drying at a temperature between about 50° C. and about 60° C.

In some embodiments, the drying occurs at a humidity less than about 30% relative humidity.

In some embodiments, the drying occurs for between about 24 hours and about 72 hours. In some embodiments, the drying occurs for about 48 hours.

In some embodiments, the drying delafloxacin meglumine trihydrate produces delafloxacin meglumine anhydrate.

In some embodiments, said further drying is under reduced pressure.

In another aspect, processes for the preparation of a crystalline anhydrous Form 1B of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate are disclosed herein, which processes comprise drying delafloxacin meglumine trihydrate under low humidity conditions.

In some embodiments, the drying occurs at a temperature between about 30° C. and about 40° C. In some embodiments, the drying occurs at a temperature of about 35° C.

In some embodiments, the drying occurs under vacuum. In some embodiments, the vacuum comprise a pressure of about 1 to about 10 mbar. In some embodiments, the pressure is about 3 mbar.

In some embodiments, the humidity is below about 30% relative humidity.

In some embodiments, the delafloxacin meglumine trihydrate is dried for between about 4 hours to about 24 hours. In some embodiments, the delafloxacin meglumine trihydrate is dried for about 12 hours.

In another aspect, processes for producing a crystalline anhydrous Form 1A of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate from a crystalline anhydrous Form 1B of D-glucitol, 1-deoxy-1-(methylamino)-, 1-(6-amino-3,5-difluoropyridin-2-yl)-8-chloro-6-fluoro-1,4-dihydro-7-(3- hydroxyazetidin-1-yl)-4-oxo-3-quinolinecarboyxlate are disclosed herein, the processes comprising exposing delafloxacin meglumine to heat and humidity.

In some embodiments, the heat is between about 30° C. and about 60° C. In some embodiments, the heat is between about 40° C. and about 50° C.

In some embodiments, the humidity is between about 20% and about 60% relative humidity. In some embodiments, the humidity is between about 30% and about 50% relative humidity.

In some embodiments, the dried delafloxacin meglumine trihydrate is exposed to heat and humidity for about 12 hours to about 48 hours.

In some embodiments, the dried delafloxacin meglumine trihydrate is exposed to heat and humidity for about 30 hours.

In some embodiments, the process further comprise drying the resulting delafloxacin meglumine to further drying.

In some embodiments, said further drying comprise drying at a temperature between about 30° C. and about 70° C. In some embodiments, said further drying comprise drying at a temperature between about 50° C. and about 60° C.

In some embodiments, said further drying occurs under reduced pressure.

In some embodiments, said further drying occurs at a humidity below about 30% relative humidity.

In some embodiments, the delafloxacin meglumine comprise delafloxacin meglumine trihydrate.

In some embodiments, the delafloxacin meglumine comprise delafloxacin meglumine anhydrate.

In some embodiments, the delafloxacin meglumine comprise a mixture of delafloxacin meglumine trihydrate and delafloxacin meglumine anhydrate.

In another aspect, a crystalline anhydrous Form 1A of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate prepared according to the processes disclosed herein is disclosed.

In another aspect, a crystalline anhydrous Form 1B of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate prepared according to the processes disclosed herein is disclosed.

### C. Detailed Description of the Figures

FIG. 1 shows an X-ray powder diffraction (XRPD) pattern of crystalline anhydrous Form 1A D-glucitol, 1-deoxy-1-(methylamino)-, 1-(6-amino-3,5-difluoropyridin-2-yl)-8-chloro-6-fluoro-1,4-dihydro-7-(3- hydroxyazetidin-1-yl)-4-oxo-3-quinolinecarboyxlate. The sample contains sodium chloride as an internal standard.

FIG. 2 shows an X-ray powder diffraction (XRPD) pattern of crystalline anhydrous Form 1B D-glucitol, 1-deoxy-1-(methylamino)-, 1-(6-amino-3,5-difluoropyridin-2-yl)-8-chloro-6-fluoro-1,4-dihydro-7-(3- hydroxyazetidin-1-yl)-4-oxo-3-quinolinecarboyxlate. The sample contains sodium chloride as an internal standard.

FIG. 3 shows a Modulated Differential Scanning Calorimetry (mDSC) thermogram of crystalline anhydrous Form 1A D-glucitol, 1-deoxy-1-(methylamino)-, 1-(6-amino-3,5-difluoropyridin-2-yl)-8-chloro-6-fluoro-1,4-dihydro- 7-(3-hydroxyazetidin-1-yl)-4-oxo-3-quinolinecarboyxlate.

FIG. 4 shows a Modulated Differential Scanning Calorimetry (mDSC) thermogram of crystalline anhydrous Form 1B D-glucitol, 1-deoxy-1-(methylamino)-, 1-(6-amino-3,5-difluoropyridin-2-yl)-8-chloro-6-fluoro-1,4-dihydro- 7-(3-hydroxyazetidin-1-yl)-4-oxo-3-quinolinecarboyxlate. This thermogram shows a non-reversible transition of Form 1B to Form 1A at about 94° C.

FIG. 5 shows an overlay of XRPD diffraction patterns of crystalline anhydrous Form 1B D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate at 30° C. at 52% Relative Humidity (RH), as well as a reference diffraction pattern for Form 1A.

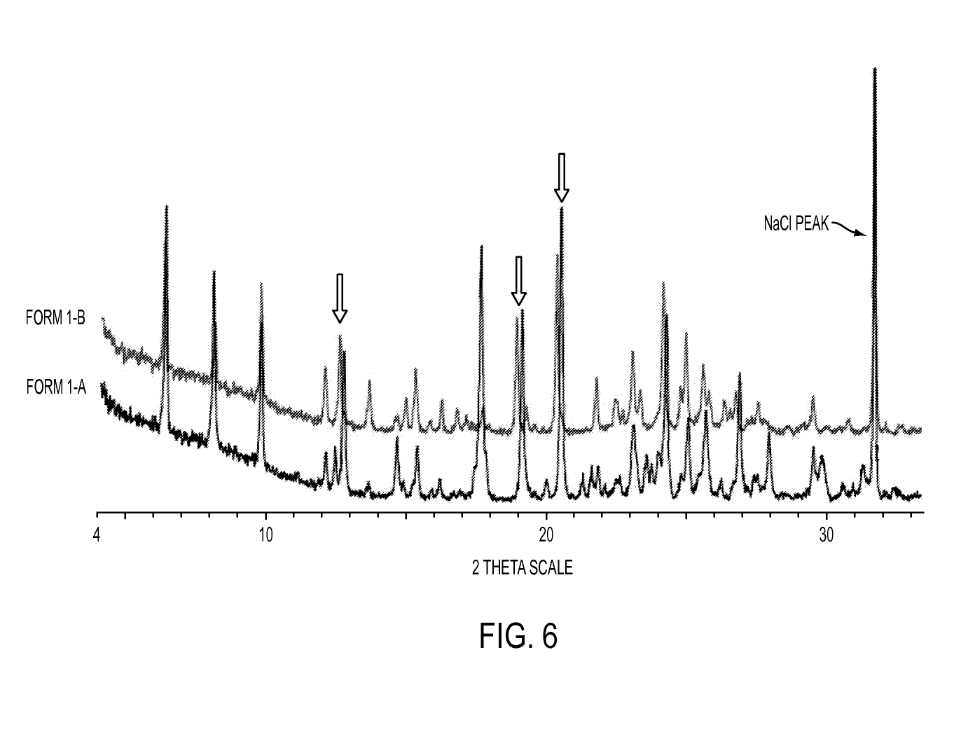

FIG. 6 shows an overlay of XRPD diffraction patterns of crystalline anhydrous Form 1A and Form 1B D-glucitol, 1-deoxy-1-(methylamino)-, 1-(6-amino-3,5-difluoropyridin-2-yl)-8-chloro-6-fluoro-1,4-dihydro-7-(3- hydroxyazetidin-1-yl)-4-oxo-3-quinolinecarboyxlate.

### Dosing

The compositions disclosed herein are useful for treating, preventing or reducing the risk of infection due to, e.g., a skin infection, nosocomial pneumonia, post-viral pneumonia, an abdominal infection, a urinary tract infection, bacteremia, septicemia, endocarditis, an atrio-ventricular shunt infection, a vascular access infection, meningitis, infection due to surgical or invasive medical procedures, a peritoneal infection, a bone infection, a joint infection, a methicillin-resistant Staphylococcus aureus infection, a vancomycin-resistant Enterococci infection, a linezolid-resistant organism infection, tuberculosis, a quinolone resistant Gram-positive infection, a ciprofloxacin resistant methicillin resistant (MRSA) infection, bronchitis, a complicated skin and skin structure infection (cSSSI), an uncomplicated skin and skin structure infection (uSSSI), a community respiratory-tract infection, and a multi-drug resistant (MDR) Gram-negative infection.

The dose of active compound and mode of administration, e.g., injection, intravenous drip, etc. will depend upon the intended patient or subject and the targeted microorganism, e.g., the target bacterial organism. Dosing strategies are disclosed in L. S. Goodman, et al., The Pharmacological Basis of Therapeutics, 201-26 (5th ed.1975), the entire contents of which is herein incorporated in its entirety.

Compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals. Furthermore, administration can be by periodic injections of a bolus, or can be made more continuous by intravenous, intramuscular or intraperitoneal administration from an external reservoir (e.g., an intravenous bag).

Where the active compound is to be used as part of a transplant procedure, it can be provided to the living tissue or organ to be transplanted prior to removal of tissue or organ from the donor. The compound can be provided to the donor host. Alternatively or, in addition, once removed from the donor, the organ or living tissue can be placed in a preservation solution containing the active compound. In all cases, the active compound can be administered directly to the desired tissue, as by injection to the tissue, or it can be provided systemically, by parenteral administration, using any of the methods and formulations described herein and/or known in the art. Where the drug comprises part of a tissue or organ preservation solution, any commercially available preservation solution can be used to advantage. For example, useful solutions known in the art include Collins solution, Wisconsin solution, Belzer solution, Eurocollins solution and lactated Ringer's solution.

In conjunction with the methods disclosed herein, pharmacogenomics (i.e. the study of the relationship between an individual's genotype and that individual's response to a foreign compound or drug) can be considered. Differences in metabolism of therapeutics can lead to severe toxicity or therapeutic failure by altering the relation between dose and blood concentration of the pharmacologically active drug. Thus, a physician or clinician can consider applying knowledge obtained in relevant pharmacogenomics studies in determining whether to administer a drug as well as tailoring the dosage and/or therapeutic regimen of treatment with the drug.

The amount administered to a patient will likely depend on such variables as the overall health status of the patient, the relative biological efficacy of the compound delivered, the formulation of the drug, the presence and types of excipients in the formulation, the route of administration, and the infection to be treated, prevented, or reducing the risk of Also, it is to be understood that the initial dosage administered can be increased beyond the above upper level in order to rapidly achieve the desired blood-level or tissue level, or the initial dosage can be smaller than the optimum.

In some embodiments, the dose of active compound comprises from about 0.1 to about 1500 mg of the compound per dose. In some embodiments, the dose of active compound is selected from about 25 mg, about 50 mg, about 75 mg, about 100 mg, about 125 mg, about 150 mg, about 175 mg, about 200 mg, about 225 mg, about 250 mg, about 275 mg, about 300 mg, about 325, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050, mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, and about 1500 mg.

As is understood by one of ordinary skill in the art, generally, when dosages are described for a pharmaceutical active, the dosage is given on the basis of the parent or active moiety. Therefore, if a salt, hydrate, or another form of the parent or active moiety is used, a corresponding adjustment in the weight of the compound is made, although the dose is still referred to on the basis of the parent or active moiety delivered. As a nonlimiting example, if the parent or active moiety of interest is a monocarboxylic acid having a molecular weight of 250, and if the monosodium salt of the acid is desired to be delivered to be delivered at the same dosage, then an adjustment is made recognizing that the monosodium salt would have a molecular weight of approximately 272 (i.e. minus 1H or 1.008 atomic mass units and plus 1 Na or 22.99 atomic mass units). Therefore, a 250 mg dosage of the parent or active compound would correspond to about 272 mg of the monosodium salt, which would also deliver 250 mg of the parent or active compound. Said another way, about 272 mg of the monosodium salt would be equivalent to a 250 mg dosage of the parent or active compound.

### Experimental

Synthesis of Crystalline Anhydrous Delafloxacin Meglumine Form 1B

Delafloxacin meglumine trihydrate (made as disclosed in International Patent Application Publication No. WO 2006/042034) was dried at 30° C. and 3 mbar for approximately 12 hours to produce the crystalline anhydrous delafloxacin meglumine Form 1B.

Process for Converting Crystalline Anhydrous Delafloxacin Meglumine Form 1B to Form 1A

A nitrogen stream was passed through a saturated solution of aqueous K₂CO₃ at a rate of about 0.5 kg/hour to maintain an outlet humidity of about 30-50% RH. The humidified nitrogen stream was passed through a preheated drier at a temperature of about 35-40° C. containing 4.55 kg of crystalline anhydrous delafloxacin meglumine Form 1B until a representative sample analyzed by XRPD showed full conversion to crystalline anhydrous delafloxacin meglumine Form 1A, after about 30 hours. The resulting cake was further dried without humidification under vacuum at about 55° C. for about 48 hours, yielding 4.52 kg of anhydrous delafloxacin meglumine Form 1A.

Synthesis of Crystalline Anhydrous Delafloxacin Meglumine Form 1A

100 kg of wet delafloxacin meglumine trihydrate (made as disclosed in International Patent Application Publication No. WO 2006/042034) was dried at about 35° C. under vacuum for 17 hours, followed by drying at about 55° C. under vacuum for 24 hours. The cake was humidified with a nitrogen stream with an inlet humidity of about 40-60% RH and a drier temperature of about 50-55° C. until a representative sample analyzed by XRPD showed full conversion to crystalline anhydrous delafloxacin meglumine Form 1A, after about 18 hours. The resulting cake was further dried without humidification under vacuum at about 55° C. for about 48 hours, yielding 86.0 kg of crystalline anhydrous delafloxacin meglumine Form 1A.

X-Ray Powder Diffraction

X-Ray Powder Diffraction patterns were collected on a Bruker AXS C2 GADDS diffractometer using Cu Kα radiation (40 kV, 40 mA), automated XYZ stage, laser video microscope for auto-sample positioning and a HiStar 2-dimensional area detector. X-ray optics consists of a single Gael multilayer mirror coupled with a pinhole collimator of 0.3 mm. A weekly performance check was carried out using a certified standard NIST 1976 Corundum (flat plate). Similar instruments can be used to obtain XRPD patterns.

The beam divergence, i.e. the effective size of the X-ray beam on the sample, was approximately 4 mm. A θ-θ continuous scan mode was employed with a sample—detector distance of 20 cm which gives an effective 2θ range of 3.2°-29.7°. The samples were exposed to the X-ray beam for approximately 120 seconds. The software used for data collection was GADDS for WNT 4.1.16 and the data were analyzed and presented using Diffrac Plus EVA v11.0.0.2 or v13.0.0.2.

Samples run under ambient conditions were prepared as flat plate specimens using powder as received without grinding. Approximately 1-2 mg of the sample was lightly pressed on a glass slide to obtain a flat surface.

Differential Scanning Calorimetry

DSC data were collected on a TA Instruments Q2000 equipped with a 50 position autosampler. Similar instruments can be used to collect DSC data. The calibration for thermal capacity was carried out using sapphire and the calibration for energy and temperature was carried out using certified indium.

Modulated temperature DSC was carried out using an underlying heating rate of 2° C./min and temperature modulation parameters of ±1.2° C. (amplitude) every 60 seconds (period). Typically 0.5-3 mg of each sample, in a pin-holed aluminum pan, was heated at 10° C./min from 25° C. to 220° C. A purge of dry nitrogen at 50 ml/min was maintained over the sample.

The instrument control software was Advantage for Q Series v2.8.0.392 and Thermal Advantage v4.8.3 and the data were analyzed using Universal Analysis v4.4A.

Intrinsic Dissolution Rate

Approximately 100 mg of the pure test compound was compressed under high pressure (7 ton), using a constructed die. No additives were added, thus avoiding any external interference in the intrinsic dissolution profile. The resulting non-disintegrating disc was transferred to the dissolution apparatus, Distek model 5100 premiere dissolution system, containing 900 mL of pH 5 buffer with 15 mM hexadecyl trimethyl ammonium bromide (HTAB), pre-heated to 37° C. The stirring speed of the paddle was set to 50 rpm. The solution was sampled at set time points with the resulting aliquots analyzed directly by HPLC, with reference to standard solutions. 5 standards, with concentrations ranging from 0.03 to 0.005 mg/mL in deionized water, and the sample solutions were injected, together with blank injections of the deionized water. The concentration was calculated using the peak areas determined by integration of the peak found at the same retention time as the principal peak in the standard injection.

Clear differences in the dissolution profiles were noted between the two phases, with significantly faster initial dissolution rates observed for crystalline anhydrous Form 1B delafloxacin meglumine samples. This was possibly attributed to almost complete dissociation to the free acid for anhydrous Form 1B samples, significantly higher than that for the crystalline anhydrous Form 1A delafloxacin meglumine samples, as confirmed by ¹H NMR analyses post IDR.

Solid State Nuclear Magnetic Resonance (NMR) Spectroscopy

Solid state ¹H NMR has shown that the unit cell of both crystalline anhydrous Form 1A and Form 1B delafloxacin meglumine contains two molecules of meglumine and two molecules of delafloxacin. Interpretation of these data show that in Form 1B one of the meglumine molecules is less constrained, while in Form 1A both meglumine molecules are ordered and identical in their confirmation and environment.

### Equivalents

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A process for the preparation of a crystalline anhydrous Form 1A of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate, which comprises the steps of:
(a) drying delafloxacin meglumine trihydrate; and
(b) exposing the dried delafloxacin meglumine to heat and humidity.

2. The process of claim 1, wherein said drying delafloxacin meglumine trihydrate is performed under reduced pressure.

3. The process of claim 1, wherein said drying delafloxacin meglumine trihydrate is performed at a temperature between about 30° C. and about 60° C.

4. The process of claim 1, wherein the delafloxacin meglumine trihydrate is dried for between about 24 hours to about 72 hours.

5. The process of claim 4, wherein the delafloxacin meglumine trihydrate is dried for about 48 hours.

6. The process of claim 1, wherein said heat is between about 30° C. and about 70° C.

7. The process of claim 6, wherein said heat is between about 50° C. and about 60° C.

8. The process of claim 1, wherein said humidity is between about 30% and about 70% relative humidity.

9. The process of claim 8, wherein said humidity is between about 40% and about 60% relative humidity.

10. The process of claim 1, wherein the dried delafloxacin meglumine is exposed to heat and humidity for between about 8 hours to about 36 hours.

11. The process of claim 10, wherein the dried delafloxacin meglumine is exposed to heat and humidity for about 18 hours.

12. The process of claim 1, further comprising drying the delafloxacin meglumine which has been exposed to heat and humidity to further drying.

13. The process of claim 12, wherein said further drying comprises drying at a temperature between about 30° C. and about 70° C.

14. The process of claim 13, wherein said further drying comprises drying at a temperature between about 50° C. and about 60° C.

15. The process of claim 12, wherein the drying occurs at a humidity less than about 30% relative humidity.

16. The process of claim 12, wherein the drying occurs for between about 24 hours and about 72 hours.

17. The process of claim 16, wherein the drying occurs for about 48 hours.

18. The process of claim 1, wherein said drying delafloxacin meglumine trihydrate produces delafloxacin meglumine anhydrate.

19. The process of claim 12, wherein said further drying is under reduced pressure.

20. A process for the preparation of a crystalline anhydrous Form 1B of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate, which comprises drying delafloxacin meglumine trihydrate under low humidity conditions.

21. The process of claim 20, wherein said drying occurs at a temperature between about 30° C. and about 40° C.

22. The process of claim 21, wherein said drying occurs at a temperature of about 35° C.

23. The process of claim 20, wherein said drying occurs under vacuum.

24. The process of claim 23, wherein said vacuum comprises a pressure of about 1 to about 10 mbar.

25. The process of claim 24, wherein said pressure is about 3 mbar.

26. The process of claim 20, wherein said humidity is below about 30% relative humidity.

27. The process of claim 20, wherein the delafloxacin meglumine trihydrate is dried for between about 4 hours to about 24 hours.

28. The process of claim 27, wherein the delafloxacin meglumine trihydrate is dried for about 12 hours.

29. A process for producing a crystalline anhydrous Form 1A of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate from a crystalline anhydrous Form 1B of D-glucitol, 1-deoxy-1-(methylamino)-, 1-(6-amino-3,5-difluoropyridin-2-yl)-8-chloro-6-fluoro-1,4-dihydro-7-(3-hydroxyazetidin-1-yl)-4-oxo-3-quinolinecarboyxlate, the process comprising exposing delafloxacin meglumine to heat and humidity.

30. The process of claim 29, wherein said heat is between about 30° C. and about 60° C.

31. The process of claim 30, wherein said heat is between about 40° C. and about 50° C.

32. The process of claim 29, wherein said humidity is between about 20% and about 60% relative humidity.

33. The process of claim 32, wherein said humidity is between about 30% and about 50% relative humidity.

34. The process of claim 29, wherein the dried delafloxacin meglumine trihydrate is exposed to heat and humidity for about 12 hours to about 48 hours.

35. The process of claim 34, wherein the dried delafloxacin meglumine trihydrate is exposed to heat and humidity for about 30 hours.

36. The process of claim 29, further comprising drying the resulting delafloxacin meglumine to further drying.

37. The process of claim 36, wherein said further drying comprises drying at a temperature between about 30° C. and about 70° C.

38. The process of claim 37, wherein said further drying comprises drying at a temperature between about 50° C. and about 60° C.

39. The process of claim 36, wherein said further drying occurs under reduced pressure.

40. The process of claim 36, wherein said further drying occurs at a humidity below about 30% relative humidity.

41. The process of claim 29, wherein the delafloxacin meglumine comprises delafloxacin meglumine trihydrate.

42. The process of claim 29, wherein the delafloxacin meglumine comprises delafloxacin meglumine anhydrate.

43. The process of claim 29, wherein the delafloxacin meglumine comprises a mixture of delafloxacin meglumine trihydrate and delafloxacin meglumine anhydrate.

44. A crystalline anhydrous Form 1A of D-glucitol, 1-deoxy-1-(methylamino)-, 1-(6-amino-3,5-difluoropyridin-2-yl)-8-chloro-6-fluoro-1,4-dihydro- 7-(3-hydroxyazetidin-1-yl)-4-oxo-3-quinolinecarboyxlate prepared according to the method of any one of claims 1 to 19.

45. A crystalline anhydrous Form 1B of D-glucitol, 1-deoxy-1-(methylamino)-, 1-(6-amino-3,5-difluoropyridin-2-yl)-8-chloro-6-fluoro-1,4-dihydro- 7-(3-hydroxyazetidin-1-yl)-4-oxo-3-quinolinecarboyxlate prepared according to the method of any one of claims 20 to 28.

46. A crystalline anhydrous Form 1A of D-glucitol, 1-deoxy-1-(methylamino)-, 1-(6-amino-3,5-difluoropyridin-2-yl)-8-chloro-6-fluoro-1,4-dihydro- 7-(3-hydroxyazetidin-1-yl)-4-oxo-3-quinolinecarboyxlate prepared according to the method of any one of claims 29 to 43.

47. A method of treating a bacterial infection in a fish or mammal in need thereof, the method comprising administering to the fish or mammal a therapeutically effective amount of a composition comprising the crystalline form of any one of claims 44 to 46.

48. The method of claim 47, wherein the composition is administered to a mammal.

49. The method of any one of claims 47 to 48, wherein the therapeutically effective amount is from about 0.03 to about 200 mg/kg body weight.
